(19) United States
(12) Patent Application Publication
(10) Pub. No.: US 2013/0131379 A1
(43) Pub. Date: May 23, 2013

(54) METHOD FOR PREPARING CARBOXYLIC ACIDS BY OXIDATIVE CLEAVAGE OF A VICINAL DIOL

(71) Applicants: Marc Lemaire, Villeurbanne (FR); Alain Favre-Reguillon, Montmerle Sur Saone (FR); Bénédicte Paquit, Roanne (FR); Sylvain Claude, Paris (FR); Yann Raoul, Crezancy (FR)

(72) Inventors: Marc Lemaire, Villeurbanne (FR); Alain Favre-Reguillon, Montmerle Sur Saone (FR); Bénédicte Paquit, Roanne (FR); Sylvain Claude, Paris (FR); Yann Raoul, Crezancy (FR)

(73) Assignees: UNIVERSITE CLAUDE BERNARD LYON I, Villeurbanne Cedex (FR); CENTRE NATIONAL DE LA RECHERCHE SCIENTIFIQUE, Paris (FR)

(21) Appl. No.: 13/582,345

(22) PCT Filed: Mar. 4, 2011
(86) PCT No.: PCT/FR2011/050456

§ 371 Date: Feb. 8, 2013

(30) Foreign Application Priority Data
Mar. 5, 2010 (FR) 1051627

(51) Int. Cl. C07C 51/377; C07C 51/31

(52) U.S. Cl. CPC C07C 51/377; C07C 51/31
USPC 562/525; 562/539

(57) ABSTRACT

The present invention relates to a method for preparing carboxylic acids, in particular mono- and dicarboxylic acids, by oxidative cleavage of a vicinal diol.

According to the invention, said method consists of reacting a vicinal diol of formula I:

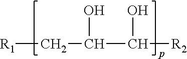

where:
p is an integer comprised between 1 and 6;
R1 and R2 are, separately:
   an alkyl or hydroxyl group having 1 to 12 carbon atoms;
   a —(CH₂)ₙ—C0₂M group where n, which can be identical or different in R1 and R2, is an integer comprised between 1 and 11 and M is a hydrogen atom or an alkyl group having 1 to 4 carbon atoms or an alkaline cation;
or R1 and R2 jointly form an alkylene —(CH₂)m- group where m is an integer comprised between 2 and 10, preferably between 2 and 6;
with industrial-grade sodium hypochlorite (or bleach), in the absence of an organic solvent and without adding a catalyst, preferably at room temperature. The invention can be used for recycling natural vegetable oils.

The present invention relates to a new method for preparing carboxylic acids, in particular mono- and dicarboxylic acids, by oxidative cleavage of a vicinal diol.

The invention notably finds application for recycling vegetable oils of natural origin, in particular oleaginous extracted oils, as well as animal oils rich in polyunsaturated fatty acids.

It is known that vegetable oils and products derived from them, such as esters and fatty acids, are commonly used as they are or as intermediates for the synthesis of numerous specialty products.

The vegetable oils constitute a source of abundantly available renewable materials and are particularly interesting because the fatty acids of which they are composed are rich in oleic acid. Thus, the products resulting from the transformation of this unsaturated acid by oxidative cleavage, namely pelargonic acid and azelaic acid, are used as precursors of esters intended for lubrication and for plasticization and as intermediates for production of polymers.

This reaction of oxidative cleavage of oleic acid can be represented by the following reaction scheme:

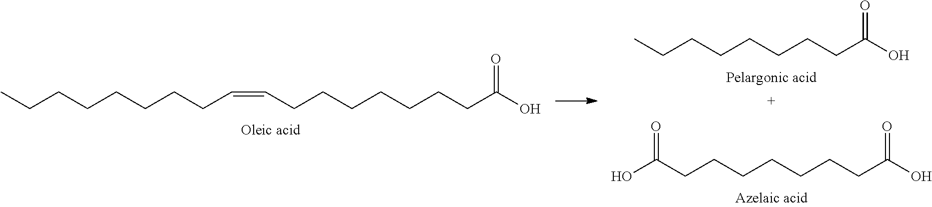 [SMILES: CCCCCCCCC(=O)O ; CCCCCCCC/C=C\CCCCCCCC(=O)O ; O=C(O)CCCCCCCC(=O)O]

Various methods for obtaining pelargonic and azelaic acids by oxidative cleavage of oleic acid are already known.

Thus, these acids can be obtained directly by ozonolysis of oleic acid (see review Rebrovic et al. Lipids Technology (1996), 135-137). This method is currently employed on an industrial scale. However, ozone is particularly difficult to use because of its hazardousness.

Catalytic methods of oxidative cleavage of oleic acid using transition metals and various oxidants (Re₂O₇/H₂O₂, H₂WO₄/H₂O₂, Ru(acac)₃/NaIO₄, RuO₂/NaOCl, RuCl₃/MeCO₃H) have been proposed in the literature. However, these methods have not been exploited industrially as they are not sufficiently competitive versus ozonolysis.

Alternative methods of preparation in two steps can also be envisaged.

A first known method consists, in a first step, of preparing 9-decenoic acid by a metathesis reaction on oleic acid, then in a second step carrying out oxidative cleavage of the terminal double bond of this acid. This oxidative cleavage is easier to carry out with the aforementioned catalytic systems.

In the context of this two-step method, the reaction scheme of which is shown below, azelaic acid and 1-decene are obtained as byproducts.

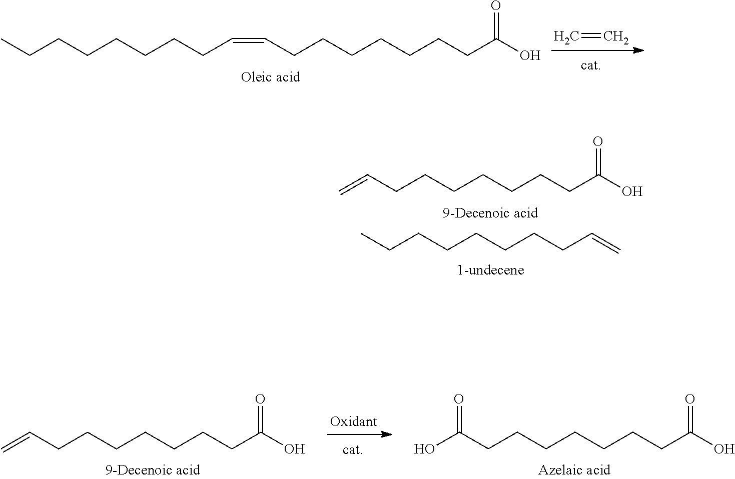

The inventors explored another method, consisting of:
in a first step, preparing a diol of oleic acid according to the methods described in the literature, for example by catalytic oxidation using hydrogen peroxide and a catalyst based on tungsten or rhenium or a carboxylic acid/H₂O₂ system as oxidant; then
in a second step, carrying out oxidative cleavage of this diol to form pelargonic and azelaic acids.

This method can be represented by the following reaction scheme:

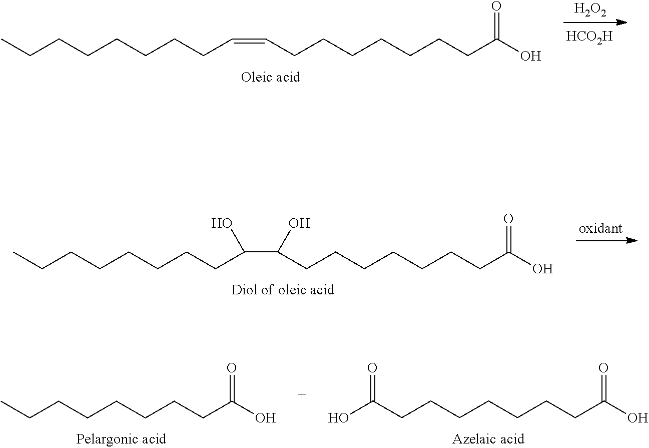 [SMILES: CCCCCCCC/C=C\CCCCCCCC(=O)O ; "HCO2H" ; "H2O2" ; CCCCCCCCC(O)C(O)CCCCCCCC(=O)O ; CCCCCCCCC(=O)O ; O=C(O)CCCCCCCC(=O)O]

According to a variant of this method, pelargonic acid and azelaic acid can be obtained by opening of the oleic acid epoxide, employing the same reaction conditions.

This variant can be represented by the following reaction scheme:

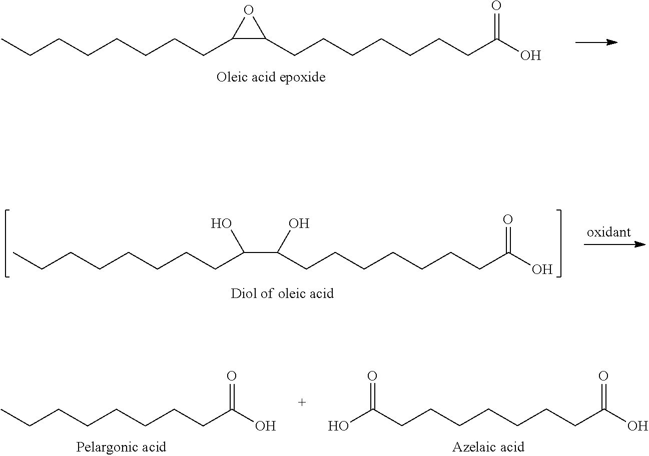

French patent 2 086 521 proposes carrying out oxidative cleavage of various vicinal diols, including the diol of oleic acid, with potassium monopersulfate and a catalytic amount of nickel (20 mol. %).

In the literature, various methods have also been proposed for carrying out the cleavage of certain diols using catalysts based on transition metals and various oxidants (Re₂O₂/H₂O₂, RuCl₃/H₂O₂, NiCl₂/NaOCl, etc.), often in an organic medium. However, these methods are rarely used for cleavage of the diol of oleic acid or of a similar diol. Moreover, the use of catalysts based on transition metals is generally to be avoided, or is even prohibited, on an industrial scale as these products, and nickel in particular, are particularly toxic.

It was discovered, and this constitutes the basis of the present invention, that it is possible to carry out oxidative cleavage of various diols, in particular diols of unsaturated carboxylic acids, such as notably oleic acid, or of derivatives thereof, for example, esters of fatty acids, in a way that is particularly simple, inexpensive, environment-friendly and does not pose any problem of safety.

Thus, according to a first aspect, the present invention relates to a method for preparing carboxylic acids, in particular mono- and dicarboxylic acids, by oxidative cleavage of a vicinal diol, characterized in that it consists of reacting a vicinal diol of formula I:

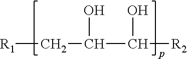

in which:
p is an integer between 1 and 6, preferably equal to 1 or 2;
R₁ and R₂ represent independently:
an alkyl or hydroxyalkyl group having from 1 to 12 carbon atoms;
a group —(CH₂)ₙ—CO₂M in which n, which can be identical or different in R₁ and R₂, is an integer between 1 and 11 and M represents a hydrogen atom or an alkyl group having from 1 to 4 carbon atoms or an alkaline cation;
   or R₁ and R₂ together form an alkylene group —(CH₂)ₘ— in which m is an integer between 2 and 10, preferably between 2 and 6;
   with sodium hypochlorite (or bleach) of industrial grade, in the absence of organic solvent and without addition of catalyst, preferably at room temperature.

As can be seen, the novelty of the method according to the invention is that it uses an oxidant that is readily available, of low cost and which is used in conditions of great safety and is environment-friendly as it is carried out in the absence of organic solvent and especially without addition of catalyst and preferably at low temperature.

In the present application, “sodium hypochlorite of industrial grade” means the commercial products usually available in the industry, having an amount of active chlorine between 10 and 20%.

“Alkyl group” means here a saturated linear or branched, preferably linear, hydrocarbon chain.

“Hydroxyalkyl group” means here an alkyl group in which at least one of the hydrogen atoms is replaced with a hydroxyl group.

According to a particular feature of the invention, the aforementioned diol corresponds to formula (I) in which:

R₁ and R₂ represent independently:
an alkyl group having from 5 to 9 carbon atoms;
a group —(CH₂)ₙ—CO₂M in which n, which can be identical or different in R₁ and R₂, is an integer between 5 and 9 and M represents a hydrogen atom or an alkyl group having from 1 to 4 carbon atoms; and p is preferably equal to 1.

The method according to the invention can be used in general for oxidative cleavage of diols derived from monounsaturated or polyunsaturated acids and derivatives thereof, such as, for example, the esters of corresponding fatty acids; in particular long-chain (notably having more than 10 carbon atoms, preferably from 10 to 30 carbon atoms), preferably obtained from a natural source and in particular from oleaginous substances, for example: soybean oil, sunflower oil, colza oil, linseed oil, olive oil, castor oil, peanut oil, palm oil, etc.

As examples of monounsaturated acid, we may mention myristoleic acid (9-tetradecenoic acid), palmitoleic acid (9-hexadecenoic acid), oleic acid (9-octadecenoic acid), ricinoleic acid (12-hydroxy-9-octadecenoic acid), gadoleic acid (11-eicosenoic acid), erucic acid (13-docosenoic acid), nervonic acid (15-tetracosenoic acid).

As examples of polyunsaturated acid, we may mention linoleic acid (9,12-octadecadienoic acid), alpha-linolenic acid (9,12,15-octadecatrienoic acid), gamma-linolenic acid (6,9,12-octadecatrienoic acid), di-homo-gamma-linolenic acid (8,11,14-eicosatrienoic acid), arachidonic acid (5,8,11,14-eicosatetraenoic acid), timnodonic acid (5,8,11,14,17-eicosapentaenoic acid), cervonic acid (4,7,10,13,16,19-docosahexaenoic acid).

This method is particularly suitable for oxidative cleavage of the diol of oleic acid (the compound of formula I above in which p is equal to 1, R₁ represents a —(CH₂)₆—CH₃ group and R₂ represents a —(CH₂)₇COOH group) into pelargonic acid and azelaic acid.

This method can also be used for oxidative cleavage of diols derived from cyclic alkenes and in particular of the diol of cyclohexene, which makes it possible to prepare adipic acid, the industrial use of which in the manufacture of Nylon is well known.

When one of the substituents R₁ and R₂ represents a group —(CH₂)ₙ—CO₂M (M representing a hydrogen atom or an alkyl group having from 1 to 4 carbon atoms and n, an integer between 1 and 11, preferably between 5 and 9) and the other represents an alkyl group having from 1 to 12 carbon atoms, preferably from 5 to 9 carbon atoms, oxidative cleavage of the compound of formula (I) leads to a mixture of monocarboxylic acid and of dicarboxylic acid, such as a mixture of pelargonic acid and azelaic acid in the case of oleic acid or leads to a mixture of monocarboxylic acid and hemiester, such as a mixture of pelargonic acid and mono-alkyl azelate in the case of an alkyl oleate.

When the groups R₁ and R₂ represent simultaneously a group of formula —(CH₂)ₙ—CO₂M (where n can be different in each of the substituents R₁ and R₂) oxidative cleavage leads to a mixture of dicarboxylic acids or even to a single dicarboxylic acid when the starting diol is symmetric, i.e. when R₁ represents a group —(CH₂)ₙ₋₁—CO₂M and R₂ represents a group —(CH₂)ₙ—CO₂M.

Thus, according to a particularly advantageous feature of the invention, the starting diol corresponds to formula (I) in which:

R₁ represents:
a group —(CH₂)ₙ₋₁—CO₂M in which n is an integer between 6 and 9 and M represents a hydrogen atom or an alkaline cation;
   R₂ represents:
a group —(CH₂)ₙ—CO₂M in which n, which is identical in R₁ and R₂, is an integer between 6 and 9 and M represents a hydrogen atom or an alkaline cation; and p is preferably equal to 1.

In the particular case when the starting diol is 9,10-dihydroxy-octadecanedioic acid, it is possible to obtain azelaic acid free from byproduct, such as pelargonic acid.

Quite surprisingly, it was shown in the context of the present invention that the vicinal diols obtained from unsaturated or polyunsaturated fatty acids can be isolated by recrystallization or by extraction with a particularly high purity, above 98%, from a natural source of these acids.

For example, 9,10-dihydroxy-octadecanedioic acid can be obtained, starting from oleic acid, by a two-step method comprising:
firstly, conversion of oleic acid to 9-octadecenedioic acid, notably employing the method of bioconversion described in international patent application WO 2006/064131, incorporated here by reference; then
secondly, carrying out the dihydroxylation of the aforementioned 9-octadecenedioic acid notably by means of a mixture of hydrogen peroxide and an organic acid such as formic acid or acetic acid, as will be described in more detail below.

According to a particularly advantageous feature of the invention, 9-octadecenedioic acid is obtained by bioconversion in which a mutant strain of Yarrowia lipolytica is submitted to a bioconversion substrate consisting of an oleic sunflower oil.

It was observed, entirely unexpectedly, that this two-step method makes it possible to obtain a product of high purity (above 98%) even when the starting product (9-octadecenedioic acid) is obtained at the end of the first step with a lower purity (for example below 80%, or even of the order of 65%).

The second step of this method can therefore be applied starting with a 9-octadecenedioic acid that is not pure, for example a commercial product with purity from 65% to 99%.

The synthesis of azelaic acid from oleic acid by the route just described can be represented by the following reaction scheme:

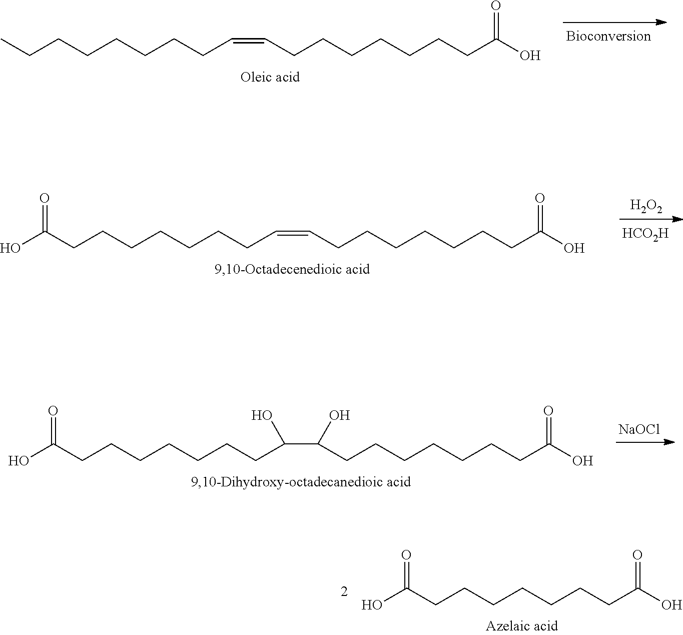

According to a particular feature of the method according to the invention, the diol of formula (I) and sodium hypochlorite are present in the reaction mixture for oxidative cleavage in a molar ratio of sodium hypochlorite to diol between 2 and 30, preferably between 3 and 5.

According to an embodiment that is currently preferred, the diol of formula (I) above is obtained by dihydroxylation of an alkene of formula (II):

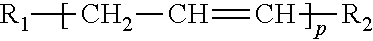

in which p, R₁ and R₂ are as defined above.

The alkene of formula (II) is in particular a monounsaturated acid or a polyunsaturated acid, preferably long-chain, as defined above.

According to another particular feature of the invention, dihydroxylation is carried out using a mixture of hydrogen peroxide and an organic acid of formula RCO₂H in which R represents a hydrogen atom or an alkyl group having from 1 to 4 carbon atoms, preferably in a molar ratio of hydrogen peroxide to organic acid between 1:5 and 1:20.

Advantageously, the aforementioned dihydroxylation is carried out using a mixture of hydrogen peroxide and formic acid or a mixture of hydrogen peroxide and acetic acid.

According to another particular feature of the invention, the dihydroxylation step is generally carried out at a temperature between 30 and 50° C. for a time from 6 to 12 h, for example at 40° C. for a time of 8 h.

The dihydroxylation makes it possible to obtain a pure dihydroxylated product from a fraction of fatty acid of natural origin.

The invention will be better understood on reading the following illustrative examples.

### EXAMPLE 1

### Dihydroxylation of Cyclohexene

4 g of cyclohexene (48.7 mmol) was added dropwise to a mixture of 6.8 mL of hydrogen peroxide at 30% v/v (68.2 mmol, 1.4 eq.) and 24 mL of formic acid (0.67 mol, 13.7 eq.) cooled to 0° C. At the end of addition, the mixture was heated at 40° C. for 8 h and then left at room temperature overnight. The water and the formic acid were then partially removed under reduced pressure until an oil was obtained. The oil was taken up in 50 mL of 1 N potassium hydroxide and heated at 90° C. for 1 h. Concentrated hydrochloric acid (37 wt. %) was added to this solution until a pH close to 2 was obtained. Cis-1,2-cyclohexanediol was obtained by decanting and then filtration, in the form of a white powder at a yield of 50% (2.83 g).

M.p.=74-76° C., FTIR analysis, ¹H and ¹³C NMR in CDCl₃ and GC/MS analysis comply with the literature (purity>99%).

### EXAMPLE 2

### Dihydroxylation of Oleic Acid at 90% Purity

50 g of oleic acid (159 mmol) of 90% purity (purity determined by GC/MS analysis of the corresponding methyl ester) was added dropwise to a mixture of 25 mL of hydrogen peroxide at 30% v/v (248 mmol, 1.4 eq.) and 91.3 mL of formic acid (2.42 mol, 13.7 eq.) cooled to 0° C. At the end of addition, the mixture was heated at 40° C. for 8 h and then left at room temperature overnight. The water and formic acid were then partially removed under reduced pressure until an oil was obtained. The oil was taken up in 150 mL of 1 N potassium hydroxide and heated at 90° C. for 1 h. Concentrated hydrochloric acid (37 wt. %) was added to this solution until a pH close to 2 was obtained. The oily phase obtained was separated using a dropping funnel and was washed with 100 mL of water. This gave 59 g of an oil.

For analytical purposes, this oil was recrystallized from hexane and the pure 9,10-dihydroxystearic acid was thus obtained in the form of a white solid.

M.p.=130-132° C., FTIR analysis, ¹H and ¹³C NMR in MeOD and GC/MS analysis of the methyl ester comply with the literature.

### EXAMPLE 3

### Dihydroxylation of 9-octadecenedioic acid at 65% by the system HCO₂H/H₂O₂/CH₂Cl₂

The 9-octadecenedioic acid used in this example is the product marketed by the company SEDERMA under the name O.D.A. (65% pure).

9-Octadecenedioic acid (65 mmol, 31.24 g), 65% pure (purity determined by GC/MS analysis of the corresponding methyl ester), was added in 1 h to a mixture of H₂O₂ 30% v/v (0.14 mol, 14.3 mL)/HCO₂H (1.4 mol, 52.8 mL)/CH₂Cl₂ (50 mL) at 0° C. At the end of addition, the reaction mixture was heated at 40° C. for 8 h and then left at room temperature overnight. The water and formic acid were partially evaporated under reduced pressure until an oil was obtained. The oil was taken up in 100 mL of 1 N potassium hydroxide and heated at 90° C. for 1 h. Concentrated hydrochloric acid (37 wt. %) was added to this solution until an acid pH was obtained. The precipitated 9,10-dihydroxyoctadecanedioic acid was recovered in the form of a white powder by simple filtration at quantitative yield (24.2 g).

M.p.=158-160° C., FTIR analysis, ¹H and ¹³C NMR in MeOD and GC/MS analysis of the methyl ester comply with the literature (purity>99%).

### EXAMPLE 4

### Dihydroxylation of 9-octadecenedioic acid at 65% by the System CH₃CO₂H/H₂O₂

9-Octadecenedioic acid (19.5 mmol, 9.37 g), 65% pure (purity determined by GC/MS analysis of the corresponding methyl ester), was dissolved in 35 mL of concentrated acetic acid. This solution was added in 30 minutes to a mixture of H₂O₂ 30% v/v (42 mmol, 9.6 mL)/CH₃CO₂H (0.42 mol, 24 mL) at 0° C. At the end of addition, the reaction mixture was heated at 40° C. for 8 h and then left at room temperature overnight. The water and acetic acid were partially evaporated under reduced pressure until an oil was obtained. The oil was taken up in 50 mL of 1 N potassium hydroxide and was heated at 90° C. for 1 h. Concentrated hydrochloric acid (37 wt. %) was added to this solution until a pH close to 2 was obtained. The precipitated 9,10-dihydroxyoctadecanedioic acid was recovered in the form of a white powder by simple filtration at quantitative yield (6.75 g).

### EXAMPLE 5

### Dihydroxylation of Methyl Oleate at 75%

The methyl oleate used in this example is a commercially available product (industrial grade).

Methyl oleate (7.5 mmol, 2.96 g), 75% pure (purity determined by GC/MS analysis), was added in 25 minutes to a mixture of H₂O₂ 30% v/v (14 mmol, 1.43 mL)/HCO₂H (0.14 mol, 5.05 mL) at 0° C. At the end of addition, the reaction mixture was heated at 40° C. for 8 h and then left at room temperature overnight. The water and formic acid were partially evaporated under reduced pressure until an oil was obtained. The oil was taken up in 50 mL of 1 N potassium hydroxide and heated at 90° C. for 1 h. Concentrated hydrochloric acid (37 wt. %) was added to this solution until a pH close to 2 was obtained. The oily phase obtained was separated using a dropping funnel and was washed with 5 mL of water.

An oil was thus obtained at quantitative yield (3.3 g).

For analytical purposes, this oil was recrystallized from hexane and pure methyl 9,10-dihydroxystearate was obtained in the form of a white solid.

M.p. 158-160° C., FTIR analysis, ¹H and ¹³C NMR in MeOD and GC/MS comply with the literature.

### EXAMPLE 6

### Oxidative Cleavage of trans-1,2-cyclohexanediol

1.16 g (10 mmol) of trans-1,2-cyclohexanediol was added to 21.6 mL (31.5 mmol) of a solution of bleach at 1.46 mol·L⁻¹ (Acros Ref 21925). After 3 h at room temperature, the aqueous phase was acidified with 37 wt. % HCl. Adipic acid was obtained by filtration at a yield of 50% (0.73 g) in the form of a white solid.

M.p. 150-152° C., FTIR analysis, ¹H and ¹³C NMR in DMSO comply with the literature.

### EXAMPLE 7

### Oxidative Cleavage of 9,10-dihydroxy-octadecanoic acid

1.58 g (5 mmol) of pure 9,10-dihydroxy-octadecanoic acid was added to 10.8 mL (15.75 mmol) of a solution of bleach at 1.46 mol·L⁻¹ (Acros Ref 21925). After 5 h at room temperature, the aqueous phase was acidified with 37 wt. % HCl. The oily phase obtained was separated using a dropping funnel and was washed with 6 mL of water. This gave 1.73 g of an oil.

For analytical purposes, the product was then dissolved in 6 mL of MeOH and then 0.6 mL of concentrated sulfuric acid was added. The reaction mixture was heated under reflux for 2 h. The product was taken up in 20 mL of water. The organic phase was extracted with 3×6 mL of hexane. The organic phase obtained was washed successively with 20 mL of water, 20 mL of sodium carbonate solution and 20 mL of water. The organic phase was dried over Na₂SO₄ and the solvent was evaporated under reduced pressure. This gave the expected mixture of esters at quantitative yield (1.94 g). After evaporation of the solvent, the product was purified by silica gel chromatography (cyclohexane/EtOAc:5/2).

Methyl pelargonate (Rf=0.84) was obtained in the form of a clear oil at a yield of 89% and dimethyl azelate (Rf=0.47) was obtained in the form of a clear oil at a yield of 40%.

¹H and ¹³C NMR analysis in CDCl₃ and GC/MS analysis of the methyl pelargonate and of the dimethyl azelate comply with the literature.

### EXAMPLE 8

### Oxidative Cleavage of 9,10-dihydroxy-octadecanedioic acid

1.04 g (3 mmol) of pure 9,10-dihydroxy-octadecanedioic acid was added to 8.6 mL (9.45 mmol) of a solution of bleach at 1.11 mol·L⁻¹ (Aldrich Ref 81070). After 5 h at room temperature, the aqueous phase was acidified with 37 wt. % HCl. Azelaic acid was obtained by decanting and then filtration and was washed with cold water.

Azelaic acid was obtained at a yield of 50% in the form of a white solid.

M.p.=107° C., FTIR analysis, ¹H, ¹³C NMR in CDCl₃ and GC/MS of the methyl diester comply with the literature.

### EXAMPLE 9

### Oxidative Cleavage of 9,10-dihydroxy-octadecanedioic acid

Pure 9,10-dihydroxy-octadecanedioic acid (2 mmol, 693 mg) was made into a paste with 3 mL of an aqueous solution of sodium azelate at 15 wt. %. 5.7 mL (6.3 mmol) of a solution of bleach at 1.11 mol·L⁻¹ (Aldrich Ref 81070) was added to this paste. After 5 h at room temperature, the aqueous phase was acidified with 37 wt. % HCl. Azelaic acid was obtained by decanting and then filtration and was washed with cold water.

Azelaic acid was obtained at a yield of 50% in the form of a white solid.

### EXAMPLE 10

### Oxidative Cleavage of methyl 9,10-dihydroxystearate

1.16 g (3.5 mmol) of pure methyl 9,10-dihydroxystearate was added to 7.5 mL (11 mmol) of a solution of bleach at 1.46 mol·L⁻¹ (Acros Ref 21925). After 3 h at room temperature, the aqueous phase was acidified with 37 wt. % HCl. The oily phase obtained was separated using a dropping funnel and was washed with 4 mL of water. This gave 1.26 g of oil.

For analytical purposes, the product thus obtained was then dissolved in 2.8 mL of MeOH, and then 0.28 mL of concentrated sulfuric acid was added. The reaction mixture was heated under reflux for 2 h. The product was taken up in 10 mL of water. The organic phase was extracted with 3×5 mL of ethyl acetate. The organic phase obtained was washed successively with 10 mL of water, 10 mL of sodium carbonate solution and 10 mL of water. The organic phase was dried over Na₂SO₄ and the solvent was evaporated under reduced pressure.

The expected mixture of esters was thus obtained at quantitative yield (1.36 g).

GC/MS Analysis (after Esterification According to the Method Described Previously) Complies with the Literature.

The examples that have just been given show that the method according to the present invention makes it possible to prepare various carboxylic acids, in particular mono- and dicarboxylic acids that are useful as synthesis intermediates for the production of polymers, such as notably azelaic acid and adipic acid.

This method is particularly advantageous, as it uses a readily available oxidant, of low cost, and which can be used in conditions of great safety and is environment-friendly.

This method is also advantageous in that the starting products can be of natural origin, such as in particular oleic acid obtained from oils extracted from oleaginous substances.

1. A method for preparing carboxylic acids by oxidative cleavage of a vicinal diol, which consists of reacting a vicinal diol of formula I: 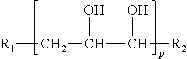
in which:
p is an integer between 1 and 6;
R₁ and R₂ represent independently:
an alkyl or hydroxyalkyl group having from 1 to 12 carbon atoms;
a group —(CH₂)ₙ—CO₂M in which n, which can be identical or different in R₁ and R₂, is an integer between 1 and 11 and M represents a hydrogen atom or an alkyl group having from 1 to 4 carbon atoms or an alkaline cation;
or R₁ and R₂ together form an alkylene group —(CH₂)ₘ— in which m is an integer between 2 and 10,
with sodium hypochlorite (or bleach) of industrial grade, in the absence of organic solvent and without addition of catalyst.

2. The method as claimed in claim 1, wherein the aforementioned diol corresponds to formula I in which:
R₁ and R₂ represent independently:
an alkyl group having from 5 to 9 carbon atoms;
a group —(CH₂)ₙ—CO₂M in which n, which can be identical or different in R₁ and R₂, is an integer between 5 and 9 and M represents a hydrogen atom or an alkyl group having from 1 to 4 carbon atoms or an alkaline cation.

3. The method as claimed in claim 1, wherein the aforementioned diol corresponds to formula I in which:
R₁ represents:
a group —(CH₂)ₙ₋₁—CO₂M in which n is an integer between 6 and 9 and M represents a hydrogen atom or an alkaline cation;
R₂ represents:
a group —(CH₂)ₙ—CO₂M in which n, which is identical in R₁ and R₂, is an integer between 6 and 9 and M represents a hydrogen atom or an alkaline cation.

4. The method as claimed in claim 2, wherein the aforementioned diol corresponds to formula I in which p is equal to 1.

5. The method as claimed in claim 3, wherein the diol of formula (I) above is 9,10-dihydroxy-octadecanedioic acid.

6. The method as claimed in claim 5, wherein 9,10-dihydroxy-octadecanedioic acid is obtained by dihydroxylation of 9-octadecenedioic acid, itself obtained from oleic acid.

7. The method as claimed in claim 1, wherein the diol of formula I and sodium hypochlorite are reacted at a molar ratio of sodium hypochlorite to diol between 3 and 30.

8. The method as claimed in claim 1, wherein the diol of formula (I) above is obtained by dihydroxylation of an alkene of formula II: 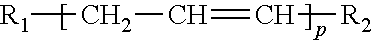
in which p, R₁ and R₂ are as defined in these claims.

9. The method as claimed in claim 8, wherein said dihydroxylation is carried out using a mixture of hydrogen peroxide and an organic acid of formula RCO₂H in which R represents a hydrogen atom or an alkyl group having from 1 to 4 carbon atoms.

10. The method as claimed in claim 9, wherein said dihydroxylation is carried out using a mixture of hydrogen peroxide and formic acid or a mixture of hydrogen peroxide and acetic acid.

11. The method as claimed in claim 1, wherein said reaction is carried out at room temperature.

12. The method as claimed in claim 3, wherein the aforementioned diol corresponds to formula I in which p is equal to 1.

13. The method as claimed in claim 6, wherein said 9-octodecanedoic acid is obtained by bioconversion of oleic acid.

14. The method as claimed in claim 1, wherein the diol of formula I and sodium hypochlorite are reacted at a molar ratio of sodium hypochlorite to diol between 3 and 5.

15. The method as claimed in claim 9, wherein said dihydroxylation is carried out at a molar ratio of hydrogen peroxide to organic acid between 1 and 20.

16. The method as claimed in claim 9, wherein said dihydroxylation is carried out at a molar ratio of hydrogen peroxide to organic acid between 1 and 5.
